(19) United States
(12) Patent Application Publication
(10) Pub. No.: US 2013/0150749 A1
(43) Pub. Date: Jun. 13, 2013

(54) PROBE FOR DIAGNOSIS AND TREATMENT OF MUSCLE CONTRACTION DYSFUNCTION

(71) Applicants: Linda B. McLean, Kingston (CA); Roy A. Young, Odessa (CA)

(72) Inventors: Linda B. McLean, Kingston (CA); Roy A. Young, Odessa (CA)

(21) Appl. No.: 13/817,845

(22) PCT Filed: Aug. 19, 2011
(86) PCT No.: PCT/CA2011/000936

§ 371 Date: Feb. 20, 2013

Related U.S. Application Data
(60) Provisional application No. 61/375,613, filed on Aug. 20, 2010.

(51) Int. Cl. A61B 5/0488

(52) U.S. Cl. CPC A61B 5/04882; A61B 5/0488
USPC 600/546

(57) ABSTRACT

A novel probe for recording EMG signals from muscles, in particular intravaginal signals from the pelvic floor muscles (PFM), is provided herein. The probe includes an insertion end having a suction head forming a vessel open at the top with attached electrodes and a distal end for attachment to a means of providing suction and an amplifier.

### CROSS-REFERENCE TO RELATED APPLICATIONS

This application claims priority to U.S. provisional application No. 61/375,613 filed Aug. 20, 2010, the entire contents of which are hereby incorporated by reference.

### FIELD OF THE INVENTION

The present invention relates to a novel probe for recording EMG signals from muscles, in particular intravaginal signals from the pelvic floor muscles (PFMs).

### BACKGROUND

Electromyography (EMG) is a tool used to record electrical voltages induced through ion shifts that occur when a muscle contracts. The arrival of an action potential at the neuromuscular junction triggers changes in muscle cell membrane permeability, eventually leading to the formation of muscle fiber action potentials. An EMG signal is a recording of all muscle fiber action potentials located within the vicinity of the detection surfaces of the particular electrodes used and is a convenient way to determine the timing and extent of neuromuscular activation. Surface EMG is the most common method used to evaluate these parameters because it is easy to use, is noninvasive and provides a signal that reflects the activity of a large number of active motor units within the muscle of interest. However, surface electrodes are generally only useful for recording the activity from muscles close to the skin's surface. Muscles that lie deep to the skin surface or to other muscles, or small muscles that run in close proximity to other muscles are best studied using more invasive approaches such as needle or fine wire electrodes.

Recording electrodes used for EMG can be placed within a muscle (e.g., via needles or fine wires), or on skin that overlays the muscle (e.g., via surface electrodes). Most EMG recordings are performed using surface electrodes oriented in a differential configuration. In this configuration, a signal recorded from one electrode is subtracted from a signal recorded from a second electrode, which is placed over the same muscle, such that any signals that are common to both electrodes are removed from the EMG signal. An advantage of this electrode configuration over a single electrode (monopolar) configuration is that it is less likely to pick up signals in its vicinity that are not generated by the muscle of interest. Such signals that are not generated by the muscle of interest are termed crosstalk. Crosstalk is most likely recorded when the electrodes are large in size (De Luca, C., 2002, Surface electromyography: Detection and recording (PDF document), retrieved from http://www.delsys.cont/KnowledgeCenter/TutorialsTechnical%20Notes.html) or if the target muscle lies in close proximity to other muscles that may be active during a given task.

The pelvic floor muscles (PFMs) are located at the pelvic outlet, in the caudal region of the bony pelvis. The PFMs serve to close this outlet, while allowing space for the urogenital and anal openings (Fritsch, H. 2006, In: Carriere B, Feldt C, eds. The Pelvic Floor. New York, N.Y.: Thieme; 1-20). These muscles primarily serve to maintain normal urinary, sexual, and ano-rectal function. The PFMs are also thought to play a role in postural control (Smith, M. D. et al., 2007, Neurourol. Urodyn., 26(3):377-85).

The pelvic floor musculature can be divided into the superficial and deep layers. The deep muscles of the pelvic floor (levator ani group and bilateral ischiococcygeus muscles) are located approximately 2.5 cm deep to the superficial perineal area (Bø, K. et al., 1988, Neurourol Urodyn., 7: 261-2). These deep PFMs are considered to be the muscles affected in many women with PPM dysfunction; thus, they are often the focus of PPM assessment and treatment by physical therapists.

The superficial muscles of the pelvic floor (bilateral ischiocavernosus and bulbospongiosus muscles, and superficial transverse perineal muscle) are located at the level of the superficial perineum (Fritsch, H. 2006, In: Carriere B, Feldt C, eds. The Pelvic Floor. New York, N.Y.: Thieme; 1-20). These muscles are responsible for closing the vaginal introitus and erecting the clitoris (Fritsch, H. 2006, In: Carriere B, Feldt C, eds. The Pelvic Floor. New York, N.Y.: Thieme; 1-20). The superficial PFMs likely play a role in sexual pain disorders (Gentilcore-Saulnier, E. et al., 2010, J Sex Med.; 7(2):1003-22; Reissing, E. D. et al., 2005, J Psychosom Obstet Gynaecol.; 26(2):107) and urinary incontinence (Mørkved, S. et al., 2004, Int Urogynecol J Pelvic Floor Dysfunct., 15(6):384-9). Despite the possible importance of the superficial PFMs in women with PPM dysfunction, commercially available intravaginal probes do not record activity from these muscles.

PFM EMG is used by urologists and neurologists to assess the reflex responses of the pelvic floor muscles to bladder filling in patients with neurologic conditions. It is used by physiotherapists and nurse specialists (e.g., continence nurse specialists) to assess the ability of their patients to contract their pelvic floor muscles (i.e., levator ani) and to provide information in regard to the patient's muscle strength or motor control (Koh, C., et al., 2008, British Journal of Surgery, 95, 1079-87; Rosenbaum, T., 2005, Journal of Sex &Marital Therapy, 31, 329-40). EMG is also used clinically to provide biofeedback during strength or motor control training.

Although fine wire (e.g., Auchincloss, C and McLean, Simultaneous recordings of surface and fine-wire pelvic floor muscle, Canadian Physiotherapy Association Annual Conference, Calgary, AB, May 28-Jun. 1, 2009) and needle (e.g., Bo, K. and Stien R., 1994, Neurourology and Urodynamics 13:35-41; Erick P. et al., Neurourology and Urodynamics, 29 (3), pp 449-457, 2010) electrodes cat be used to record EMG from the PFMs, surface EMG is preferable as it is less invasive, and can adequately access the PFMs through the walls of the vaginal and/or anal canals. Both of these environments are moist in nature. As a result, design of adhesive surface electrodes that are commonly used for EMG recordings of other skeletal muscles is not appropriate. For example, for EMG of an arm muscle, an adhesive electrode can adhere to the skin of the arm, but such adhesion is ineffective in a moist mucus membrane environment. Instead, electrodes that are mounted onto a probe's surface are typically used. The probe is inserted into the patient's vagina or anus and surface EMG of the pelvic floor muscles are recorded (Bo, K., & Sherburn, 2005, Physical Therapy, 85, 269-82).

Drawbacks with currently available technology include that most probes use a monopolar electrode configuration with either large circumferential electrodes encircling the probe or one electrode on each side of the probe. These large electrodes and their configuration make them very susceptible to crosstalk (van der Velde, J., & Everaerd, W., 1999, International Urogynecology Journal, 10, 230-6; Madill, S., & McLean, 2004, Proceedings from the International Society of Electrophysiology and Kinesiology (ISEK) conference, Boston Mass., June 18-21; Peschers, U., et al., 2001, International Urogynecology Journal, 12, 27-30). A likely source of such crosstalk is obturator internus muscle since it shares its medial border with the pelvic floor muscles (Schunke, M. et al., 2006, Thieme Atlas of Anatomy: General Anatomy and Musculoskeletal System. Stuttgart, Germany: Thieme). Currently available probes have electrode configurations that do not allow for different pelvic floor muscles on each side of vaginal canal (levator hiatus) to be investigated or presented separately to the patient for assessment or biofeedback training.

The probes, being rather large, can also be uncomfortable, especially if they are used to record activity when the user changes positions or performs a functional activity (Brown, C., 2007, Reliability of Electromyography Detection Systems for the Pelvic Floor Muscles, retrieved from http://hdl.handle.net/1974/948). Deformation caused by a functional activity may alter the contractile characteristics of the underlying pelvic floor muscle (Morin, M. et al., 2004, Neurology and Urodynamics, 23, 668-74). In addition, there is little to no control over where the electrodes sit with respect to the location of the pelvic floor muscles in a given user when the probe is inserted into the vagina. Voorham-van der Zalm et al. (Voorham-van der Zalm, P. et al., 2006, Acta Obstetricia & Gynecologica Scandinavia, 7, 850-55) found that the electrodes on the Periform™ (NEEN Mobilis Healthcare Group, Lancashire, United Kingdom) and Veriprobe™ (Verity Medical Ltd., Hampshire, United Kingdom) do not match the location of the pelvic floor muscles.

Electromyographic (EMG) signals can also be contaminated by motion artifact, affecting the signal's validity. Motion artifact occurs when the recording electrode(s) moves along the skin surface, or the skin below the electrode is deformed or stretched, altering the voltage being detected by the electrode. In many areas of the body, motion artifact is reduced by securing the electrode to the skin with adhesives and by using a recessed electrode with a conductive medium between the electrode and the skin surface. However, when recording surface EMG from the pelvic floor muscles (PFMs), electrodes need to be located within the vagina, located at the level of the PFMs that lie adjacent to the vaginal wall. The electrodes used for this purpose are often stainless steel bars mounted on intravaginal probes. The moist environment of the vagina does not allow for electrodes to be adhered to the vaginal wall, thus most intravaginal electrodes are prone to motion artifact. Given the rigid nature of a vaginal probe, it is suspect to much movement if users perform functional activities or tasks. In particular, electrodes mounted on intravaginal probes are subject to motion artifact during actions such as coughing, laughing or sneezing, in which an abrupt and strong increase in intra-abdominal pressure generates a caudal force at the level of the probe. The electrode may also become partially or completely expelled from the vagina.

In sum, commercially available intravaginal probes possess deficiencies in their design such as problems with probe geometry, electrode size, location, and/or configuration. It would be desirable to be provided with improved EMG probes for use in research and clinical practice which overcome at least some of these deficiencies, such as probes that minimize the stretch of the PFMs, employ small electrode surfaces that are close together and provide differential signals, and/or do not move with respect to the vaginal wall.

### SUMMARY OF THE DESCRIPTION

In one aspect, there is provided herein a probe for electromyography, comprising a bowl-shaped portion at an insertion end of the probe; at least two electrodes disposed substantially on a rim of the bowl-shaped portion; and at least two wires, each connected at a first end to a said electrode and suitable for connection to an electronic device at a second end; wherein the bowl-shaped portion is attachable via suction to a membrane such that the electrodes contact the membrane and an electromyography signal is produced in said wires.

In another aspect, the probe further comprises a fitting at a distal end of the probe, the fitting having a closed position and an open position such that suction may be applied or released when the fitting is in the open position and suction may be maintained when the fitting is in the closed position.

In a further aspect, there is provided herein a method of electromyography, comprising placing the probe described herein at a location for electromyographical study; applying suction so that the bowl shaped portion attaches to a membrane; and measuring an electromyography signal. In another aspect, there is provided a method of obtaining an electromyography signal, comprising placing the probe described herein at a location for electromyographical study; and applying suction so that the bowl shaped portion attaches to a membrane; wherein an electromyography signal is obtained from said wires. In an aspect, once suction is applied and maintained, the bowl-shaped portion is substantially fixed at a position on the membrane.

In yet another aspect, the probe or method described herein is used to conduct electromyography in respect of one or more muscles accessible via a membrane of a body cavity. The body cavity may be, for example, the vagina, the rectum, the colon, the mouth, the nostril or the alimentary canal.

In an aspect, there is provided herein a probe for electromyography, comprising an insertion end for attachment to a membrane and a distal end for connection to a means for providing suction and for attaching the electrode wires or leads to an amplifier system. The insertion end has a shaped portion which forms a vessel open at the top; at least two electrodes attached to the shaped portion; and at least two wires, each wire connected at a first end to an electrode and suitable for connection to an electronic device at a second end. The insertion end is attachable via suction to the membrane such that the electrodes contact the membrane and an electromyography signal is recorded from muscles accessible via the membrane.

In another aspect, the insertion end of the probe further comprises a connector arm for attachment to a catheter, the connector arm being attached to the shaped portion. The connector arm may be connected to a catheter at a first end, and at least two wires are then housed inside the central longitudinal cavity of the catheter and exit the catheter at a second end. In an embodiment, the second end of the catheter may be attached to a means for providing suction, such as a syringe or a pump. In another embodiment, the second end of the catheter may be attached to a first end of a hollow connector having a longitudinal central cavity, with a second end of the hollow connector attached to a means for providing suction, such as a syringe or a pump. In yet another embodiment, the second end of the hollow connector is attached to a first end of a fitting having a hollow central longitudinal core that can be in an open or a closed position, and a second end of the fitting is attached to a means for providing suction. In a particular embodiment, the fitting is a stopcock.

In a further aspect, the at least two electrodes are disposed substantially at or on the watts of the shaped portion. The at least two electrodes may be, for example, bent over the wall of the shaped portion, located within the top of the wall of the shaped portion, or encircled by a round fitting attached to the wall of the shaped portion. The fitting attached to the wall of the shaped portion may be made of plastic, or the same material of which the shaped portion is made, or any other suitable material.

In an embodiment, the at least two electrodes are located at or near the top of the wall of the shaped portion. In another embodiment, the at least two electrodes are located below the top of the wall of the shaped portion. For example, the at least two electrodes may be located at about 1 mm, or between about 0.5 mm to about 3 mm, below the top of the wall of the shaped portion.

In an embodiment, the diameter of the shaped portion is about 7 mm, about 10 mm, or between about 9 mm and about 12 mm. In an aspect, therefore, the distance between the at least two electrodes is about 7 mm, about 10 mm, between about 9 mm and about 12 mm, or between about 7 mm and about 10 mm.

In one embodiment, the walls of the shaped portion are about 10 mm to about 12 mm high.

In an embodiment, the diameter of the vessel formed by the shaped portion is about 7 mm, about 10 mm, or between about 9 mm and about 12 mm. In an aspect, therefore, the distance between the at least two electrodes is about 7 mm, about 10 mm, between about 9 mm and about 12 mm, or between about 7 mm and about 10 mm.

In an embodiment, the at least two electrodes are attached to or bent over an inner ring which is placed inside the wall of the shaped portion. The inner ring may be fixed in place, for example using an adhesive such as epoxy.

In an embodiment, the distance between the at least two electrodes is about 7 mm, about 10 mm, between about 7 mm and about 10 mm, or between about 5 mm and about 12 mm.

In an aspect, the insertion end of the probe is attached to the vaginal membrane and the muscles for which EMG is recorded are pelvic floor muscles. In other aspects, the membrane to which the probe is attached is in the rectum, the colon, the mouth or the alimentary canal.

In yet another aspect, there is provided herein a probe for electromyography, comprising an insertion end for attachment to a membrane, and a distal end for connection to a means for providing suction and for attaching the at least two wires to an amplifier system. The insertion end comprises: a shaped portion which forms a bowl-shaped vessel open at the top and having a diameter of about 10 mm; at least two electrodes attached to the shaped portion, wherein the at least two electrodes are encircled by a round wall or fitting whose edge is flush with the wall of the shaped portion, and the electrodes are located at about 1 mm below the top of the shaped portion; at least two wires, each wire connected at a first end to one of the electrodes and suitable for connection to an electronic device such as an amplifier or pre-amplifier inputs at a second end; and a connector arm for attachment to a catheter, the connector arm being attached to the shaped portion, wherein the connector arm is at approximately the 6 o'clock position and the at least two electrodes are at approximately the 3 and 9 o'clock positions. The connector arm is connected to the catheter at a first end, and the at least two wires are housed inside the central longitudinal cavity of the catheter and exit the catheter at a second end. The distal end of the probe comprises: the second end of the catheter, which is attached to a first end of a hollow connector having a longitudinal central cavity, where a second end of the hollow connector is attached to a stopcock, with the stopcock attached to the means for providing suction. The insertion end of the probe is attachable via suction to the membrane such that the electrodes contact the membrane and an electromyography signal is recorded from muscles accessible via the membrane. In an embodiment, the means for providing suction is a syringe. In another embodiment, the means for providing suction is a pump. In one embodiment, the round wall or fitting whose edge is flush with the wall of the shaped portion is made of plastic.

In some embodiments, the probe is disposable. In further embodiments, the probe is sterilizable and can be reused, i.e., used more than once.

There are also provided herein methods for performing electromyography, using the probe of the invention. For example, the probe may be placed at a location for electromyographical study; suction is applied so that the insertion end of the probe attaches to a membrane; the wires at the distal end of the probe are attached to an amplifier system; and an electromyography signal is measured.

In another aspect, there is provided a method for performing electromyography, comprising placing the probe described herein at a location for electromyographical study; applying suction so that the insertion end attaches to a membrane; attaching the wires to an amplifier system; and measuring an electromyography signal. There is further provided a method of obtaining an electromyography signal, comprising placing the probe described herein at a location for electromyographical study; applying suction so that the insertion end attaches to a membrane; and attaching the wires to an amplifier system; wherein an electromyography signal is obtained from said wires.

In an aspect, once suction is applied and maintained, the insertion end is substantially fixed at a position on the membrane. In another aspect, electromyography is conducted in respect of one or more muscles accessible via the membrane of a body cavity. The body cavity may be, for example, the vagina, the rectum, the colon, the mouth, the nostril or the alimentary canal. In an aspect, electromyography is conducted in respect of the pelvic floor muscles.

In a particular aspect, the electrodes are aligned along the anteroposterior axis of a subject when the insertion end is attached to a membrane. When the membrane is the vaginal membrane and pelvic floor muscles (PFMs) are measured, the electrodes are aligned along the anteroposterior axis of the subject and/or are aligned with the PFM muscle fibers.

For the probes and methods described herein, when the electrodes are recessed, i.e., located below the top of the wall of the shaped portion, conductive paste may be applied to the electrodes before the probe is placed in position on a membrane.

In yet another aspect, there is provided herein a method for performing electromyography of pelvic floor muscles in a subject, comprising placing the probe described herein on a vaginal membrane; applying suction so that the insertion end attaches to the membrane; attaching the wires to an amplifier system; and measuring an electromyography signal; wherein the insertion end is attached to the membrane such that the electrodes are aligned along the anteroposterior axis of the subject or are aligned with the PFM fibers.

### BRIEF DESCRIPTION OF THE DRAWINGS

Particular embodiments of the present invention will now be explained by way of example and with reference to the accompanying drawings, in which:

FIG. 1 shows a schematic diagram of an embodiment of the insertion end of a probe of the invention (“Probe 1”); left: top view, right: side view.

FIG. 2 shows photographs of an embodiment of Probe 1, which is diagrammed schematically in FIG. 1, wherein in (A) is shown a photograph of the insertion end (suction head assembly), and in (B) is shown a photograph of the distal end (distal assembly), where wires are fed through catheter tubing and are connected to an amplifier system; a syringe is used to withdraw air from the conduit as the probe is placed in situ such that the suction head adheres to the tissue.

FIG. 3 shows a schematic diagram of an embodiment of the insertion end of a probe of the invention (“Probe 2”); left: top view, right: side view.

FIG. 4 shows a schematic diagram of an embodiment of the insertion end of a probe of the invention (“Probe 3”); left: top view, right: side view.

FIG. 5 shows a schematic diagram of an embodiment of the insertion end of a probe of the invention (“Probe 4”); left: top view, right: side view.

FIG. 6 shows a schematic diagram of several different embodiments of the suction head of probes of the invention, wherein different suction head configurations are shown, corresponding to Probes 1, 2, 3 and 4 as indicated (top views are shown); at the top left of the figure, a schematic diagram of an embodiment of a probe of the invention is shown.

FIG. 7 shows the effect of isolated right hip adductor contractions on the EMG signal recorded at the right PFMs while women attempt to keep their PFMs relaxed using two different electrodes: an embodiment of the invention (Probe 1; light grey) and the Femiscan™ probe (Mega Electronics Ltd., Kuopio, Finland) (dark grey) for twenty healthy females. The smoothed EMG amplitude is shown on the Y axis, and the intensity of hip contraction is shown on the X axis. Note that when the Femiscan™ probe is used, there is a significant (p>0.05) increase in EMG activity recorded from the PFMs at all levels of hip adduction contraction (25%, 50% and 100% of maximum voluntary hip adduction contraction), but when the invention (Probe 1) is used, EMG activity does not increase at the PFM electrode (p<0.05 at 25 and 50% maximum voluntary hip adduction contraction) until a maximal hip adduction contraction is performed (p>0.05). This result suggests that the Femiscan™ is picking up crosstalk at tower levels of contraction. Since both electrodes pick up significantly more EMG activity at the PFMs when a maximal hip adduction contraction is performed, it is not possible to tell in this case whether the increase in activity at this level is crosstalk or co-activation of the pelvic floor muscles.

FIG. 8 similarly shows the effect of hip adductor contractions on activity recorded from the PFMs while 20 women perform a maximal PFM contraction combined with graded hip adductor contractions using two different electrodes: an embodiment of the invention (Probe 1; light grey) and the Femiscan™ probe (dark grey). The smoothed EMG amplitude is shown on the Y axis, and the intensity of hip contraction is shown on the X axis.

FIG. 9 shows the effect of isolated right hip external rotator contractions on the EMG signal recorded at the right PFMs while women attempt to keep their PFMs relaxed using two different electrodes: an embodiment of the invention (Probe 1; light grey) and the Femiscan™ probe (dark grey) for twenty healthy females. The smoothed EMG amplitude is shown on the Y axis, and the intensity of hip contraction is shown on the X axis. Note, as with the hip adductor contractions, that when the Femiscan™ probe is used, there is a significant (p>0.05) increase in EMG activity recorded from the PFMs at all levels of hip external rotation contraction (25%, 50% and 100% of maximum voluntary hip external rotation contraction), but when the invention (Probe 1) is used, EMG activity does not increase at the PFM electrode (p<0.05 at 25 and 50% maximum voluntary hip external rotation contraction) until a maximal hip external rotation contraction is performed (p>0.05). This result suggests that the Femiscan™ is picking up crosstalk at tower levels of contraction. Since both electrodes pick up significantly more EMG activity at the PFMs when a maximal hip external rotation contraction is performed, it is not possible to tell in this case whether the increase in activity at this level is crosstalk or co-activation of the pelvic floor muscles.

FIG. 10 shows the proportion of files recorded by each electrode for which a motion artifact was identified during a coughing task. Both embodiments of the probe of the invention (Probes 1 and 4) performed significantly better than the Femiscan™ probe (Femiscan) at minimizing motion artifact; * indicates a significant difference (p<0.05) from the Femiscan™ probe. The Femiscan™ probe and Probe 1 were tested on the same sample of 18 women with no history of pelvic floor muscle disorders while they performed three repetitions of a maximal effort cough. Another embodiment of the invention (Probe 4) was subsequently tested on a sample of 15 women with stress urinary incontinence while they performed three repetitions of a maximal effort cough.

FIG. 11 shows results from a crosstalk study using Probe 4. Three women participated in this study. Fine wire electrodes were placed in the right pelvic floor muscles (top panel), the right obturator internus muscle (second panel) and an embodiment of Probe 4 was inserted and adhered to the vaginal wall at the level of the pelvic floor muscles on both the left (third panel) and right (bottom panel) sides. This figure depicts the electromyography (EMG) data recorded simultaneously from all electrodes during a moderately strong contraction of the hip external rotators. The arrow indicates the onset of obturator internus muscle activity during the hip external rotation contraction, it is evident in this figure that the obturator internus muscle is activated in isolation of the pelvic floor muscles, and that the embodiment of Probe 4 has not recorded any crosstalk from the obturator internus muscle.

### DETAILED DESCRIPTION

There is provided herein a novel probe for recording electromyographic (EMG) signals from muscle. The probe described herein has been designed based on several principles for optimizing the quality of the recorded EMG data. For example, the probe described herein may allow EMG recordings and electrical stimulation at a specific and localized muscle, minimize crosstalk, minimize motion artifact, improve the signal to noise ratio, and/or provide improved comfort for the user, compared to other probes currently in use.

The probe uses reversible suction to temporarily adhere to a moist mucous membrane such as a vaginal wall or a large intestine wall. In addition, the electrodes are placed relatively close together, compared to other probes known in the art. In one aspect, the close relative position of the electrodes minimizes crosstalk. In another aspect, adhesion of the electrodes to the tissues via suction prevents functional activities from causing motion artifact.

Where the same reference numbers are used herein in different embodiments and figures, they refer to like parts.

In some embodiments, the probe has two ends, an insertion end 20 and a distal end 21. The insertion end 20 includes a suction head assembly 4 containing electrodes 2. The suction head 4 has an attached connector arm 1 for connection to a catheter 12 in which electrode leads or wires 13 are located. The electrode leads or wires can then be connected at the distal end to any amplifier system using standard means, e.g., alligator clips.

The suction head 4 assembly includes a shaped portion, with connector arm 1 attached to it. The shaped portion comprises walls 10 that surround an opening 14, e.g., a round opening; in other words, the shaped portion forms a vessel, i.e., a hollow cavity or container, which is open at the top. It will be understood that the shape of the shaped portion and opening, in other words the vessel or cavity, may vary depending on the materials and methods of construction which are used. It may be round or substantially round bowl-shaped), oval or substantially oval, rectangular, and so on, as long as the shape allows for two electrodes to be placed on the walls substantially opposite from each other and for adherence onto a desired location.

The catheter 12 may be of any type of tubing which is strong enough to maintain some suction (i.e., vacuum) without collapsing. For example, flexible plastic tubing or catheter tubing may be used. The length of the catheter will vary depending on the location of the muscles being tested, the tests being performed, the length required to allow connection to an amplifier system, and other practicalities, which wilt be readily appreciated by the practitioner. Typically, the catheter is about 30 cm in length, or about 5 cm about 10 cm, about 20 cm, about 40 cm, about 50 cm, or about 60 cm in length. In the case of measuring PFMs, the catheter tubing should be long enough to exit the vagina.

The interior diameter of the catheter is typically about 3 mm to about 4 mm. It should be understood that any catheter tubing may be used, as tong as the opening is wide enough to allow passage of two wires and the tubing is strong enough to maintain some suction (i.e., vacuum) without collapsing. In an embodiment, the catheter is silicone tubing.

The walls 10 of the shaped portion house the electrodes. The electrodes may be attached to the walls of the shaped portion in a variety of ways, so tong as they are held in place and positioned so as to make the desired contact with the tissue. For example, the electrodes may be bent over the wall; encircled by a round fitting, optionally of plastic or another suitable material; located in a well; recessed within the top of the walls (e.g., located between the outer rim 5 and the inner rim 7 of the shaped portion (where 5 is the outer rim of the wall of the shaped portion and 7 is the inner rim of the wall of the shaped portion)); located at or on the top of the wall; or located below the top of the wall. Other configurations are possible, as long as the electrodes are held firmly in place; are substantially level with each other (located substantially in the same plane relative to the top of the suction head assembly); and are at approximately opposite sides of the opening from each other. In some configurations, the electrodes may be bent or looped over an inner ring 8, which is then placed inside the wall 10 of the shaped portion. In an embodiment, when an inner ring is placed inside the shaped portion, it may then be fixed in place (to secure the ring and the attached electrodes in place), e.g., with epoxy, polyurethane adhesive or another suitable adhesive.

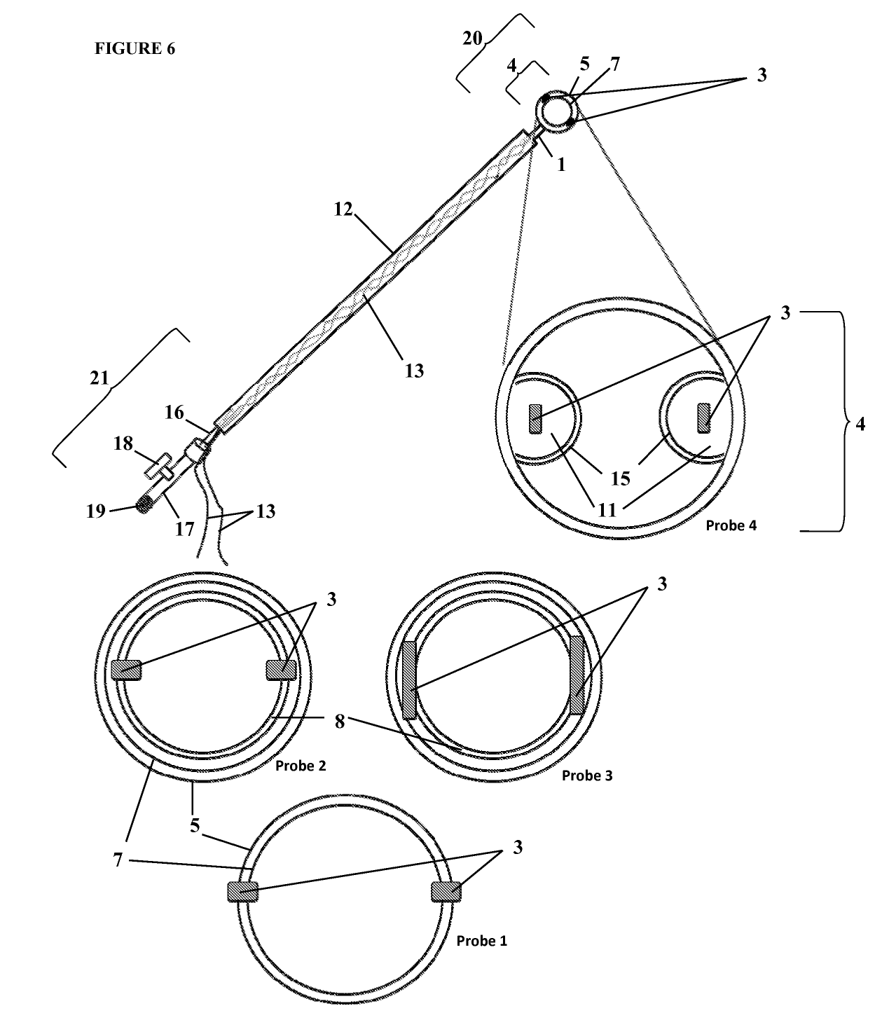

Several configurations are shown herein, e.g., in FIG. 6. It will be appreciated by the skilled artisan that many other configurations are possible.

For the purposes of placing the probe in the vagina to measure PFMs, the electrodes should be placed approximately in line with the anteroposterior axis of the subject (that is, approximately perpendicular to the cephal-caudal axis of the subject), in order to allow proper alignment with the axis of PFM contraction. Thus, if the location of the connector arm 1 is considered to be 6 o'clock, the electrodes will typically be placed at approximately the 3 and 9 o'clock positions relative to the connector arm. It should be understood that other electrode configurations are possible. For example, the electrodes may be placed between about 2 and 4 o'clock on one side and between about 8 and 10 o'clock on the other side, e.g., approximately at 2 and 9 o'clock, 2 and 8 o'clock, 3 and 10 o'clock, 4 and 10 o'clock, and so on, as long as the electrodes are located one on each side of the opening or substantially opposite each other, and are generally aligned in series along the line of action of the muscle of interest. For example, the electrodes should be generally aligned with the anteroposterior axis of the subject when placed in the vagina.

Each electrode is operationally connected to an electrical wire 13 that runs the length of the catheter 12 and that is housed inside the central longitudinal cavity of the catheter. The wires exit the catheter at its distal end and can then connect to a variety of pre-amplifier inputs (e.g., via any conventional means such as snap fastener, alligator clip, etc).

The distal end of the catheter is also connected to a hollow connector 16 having a longitudinal central cavity. This connector has a first end that is attached (e.g., frictionally connected) to the catheter and a second end that is attached to an apparatus for providing suction. Any device or means for applying suction in a consistent, controlled and releasable fashion may be used. For example, the second end of the connector may be attached to a syringe, a pump, etc.

In another embodiment, the second end of the connector 16 is attached to a fitting 17 having a hollow central longitudinal core that can be in an open position or a closed position, i.e., it can be reversibly closed off, and the fitting may then be attached to a means for applying suction. In the embodiment shown in FIG. 6, this reversible closing off of the fitting is performed using a stopcock 18 that is located at the side of the fitting 17 in-between its ends. Any other suitable means for reversibly or releasably closing off the connector or the catheter may be used.

In an embodiment, the fitting's distal end has a port 19 that is suitable to receive a syringe. For example, the syringe may screw into the port or may be inserted and retained using friction. In practice, suction may then be created by using the syringe to draw air from the probe, effectively creating a vacuum which holds the suction head 4 in place. It will be understood that suction can be released, for example, by moving the plunger in the syringe back to its original position.

To use the probe, the suction head is placed on the muscles or skin covering the muscles. The suction head is pressed into the tissue wall at the desired location. While holding the suction head in position, the operator applies suction (e.g., by drawing back on a syringe fastened to the distal end of the catheter) which creates a suction force that holds the suction head and attached electrodes in place. Once sufficient suction is applied such that the electrode is securely held in place, the operator closes off the catheter to maintain the suction. For example, if there is a stopcock placed between the connector and the syringe, then the stopcock is turned to the closed or off position, to maintain the suction. When data collection is complete, the suction head and electrodes are easily withdrawn by releasing the suction, for example by opening the stopcock, and tugging on the catheter.

In embodiments where the electrodes are located or recessed below the top of the wall of the shaped portion, it may be necessary or desirable to fill the recessed electrode cavities with a conductive paste before putting the suction head in place. Since the electrodes are located below the top of the wall of the shaped portion, the conductive paste will contact the tissue and ensure good conduction to the electrodes. Any suitable conductive paste or material that is biocompatible, of which many are known in the art, can be used.

The electrodes may be located below the top of the wall of the shaped portion at about 1 mm below the top of the wall, suction head, or vessel. In other embodiments, the electrodes may be located or recessed below the top from about 0.5 mm to about 3 min, or from about 1 min to about 3 mm, or at about 0.5 mm, at about 0.75 mm, at about 1 mm, at about 1.25 mm, at about 1.5 mm, at about 1.75 mm, at about 2 mm, at about 2.5 mm, or at about 3 mm. In another embodiment, the electrodes are located or recessed about 0.040 inches below the top of the wall of the shaped portion, suction head, or vessel. In another embodiment, the electrodes are located or recessed about 0 mm, i.e., the electrodes are not lowered or recessed relative to the top of the suction head, the wall of the shaped portion or the vessel.

In an embodiment, to record EMG signals from the PFMs, the suction head is inserted into the vagina using a gloved finger (after filling the electrode cavities with conductive paste, in the case where the probe has lowered or recessed electrodes). The operator palpates the PFMs (approximately 2.5 cm beyond the entrance to the vagina) and presses the electrode head into the tissue wall at that location. While holding the electrode head in position, the operator draws back on a syringe fastened to the distal end of the catheter, which creates a suction force that holds the electrode head onto the vaginal wall. Once sufficient suction is applied such that the electrode is securely fastened, the operator closes off the catheter to maintain the suction, e.g., closes off a stopcock, and then withdraws his/her finger, leaving the electrode in situ. A separate probe can be situated on each side of the vaginal wall to record separate EMG signals from the right and left PFMs. When data collection is complete, the suction heads/electrodes are easily withdrawn by opening the stopcock to release the suction and tugging on the catheter leading to each electrode.

The amount of suction to be applied will depend on the muscle being studied and its location. It will be understood by the practitioner that sufficient suction is required to hold the probe in place without creating undue pressure which causes discomfort to the user or injures the underlying tissues. Typically, approximately 1 cc of air is withdrawn from the syringe, which results in an increase in suction force of approximately 50 kPa. In other embodiments, a suction force of approximately 30 kPa to approximately 60 kPa is used. The suction force to be used will depend on several factors, such as the thickness of the tissue wall to which the electrode is being adhered, the activity being done by the subject during the measurement and the muscles being measured.

The embodiments shown herein use stainless steel electrodes; however it is intended that any suitable conductive material may be used. Non-limiting examples of such materials which can be used to make electrodes include silver, gold, silver chloride, platinum, nickel, nickel alloy, graphite, low alloy, aluminum, copper, copper alloy, steel, titanium and tungsten.

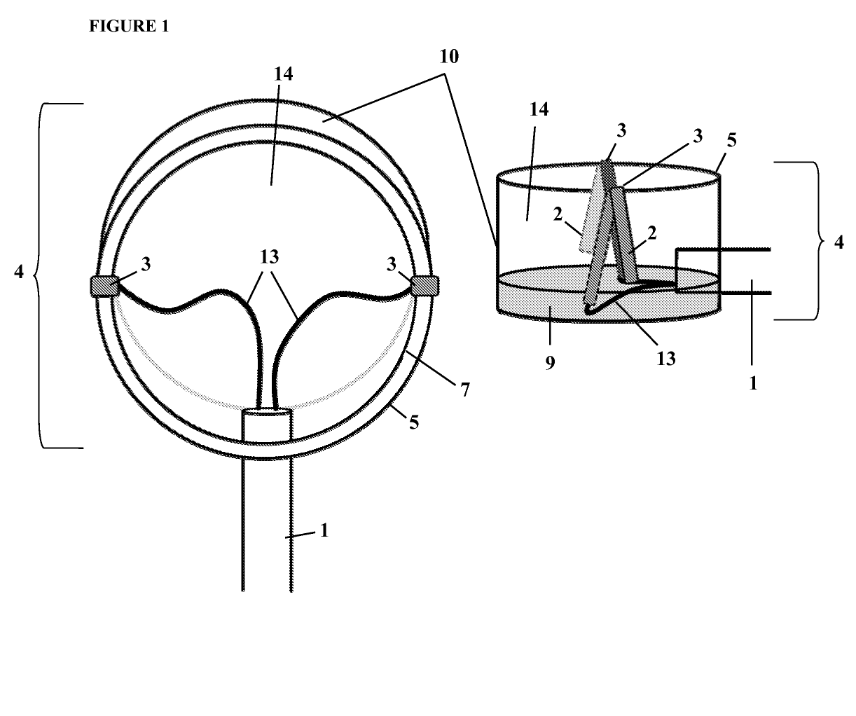

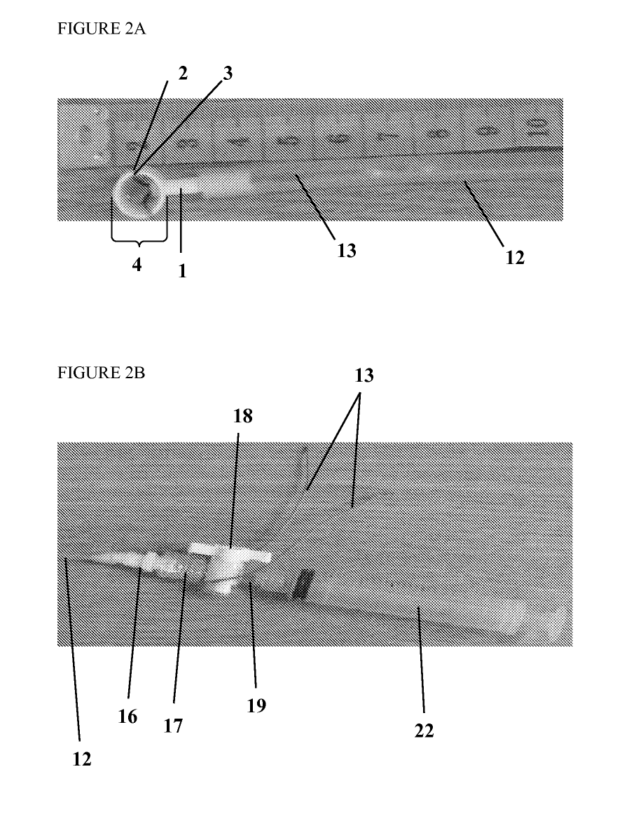

A first embodiment of the probe described herein is shown in FIGS. 1 and 2 (hereinafter referred to as “Probe 1”). In this embodiment, a round suction head 4 that is 7 mm in diameter has a stainless steel electrode 2 (approx. 1 mm² in area) located on each side (at the 3 o'clock and 9 o'clock positions), with the electrode tip or detection surface 3 located flush with, or slightly raised above, the top of the suction head 4. The electrodes 2 are made from stainless steel wires bent over the suction head edge 5 such that approximately 1 mm² of the wire (the detection surface 3) is in contact with the vaginal wall when the probe is in situ. The bottom of the suction head 4 is filled with epoxy 9 setting the wires and detecting surface in place (seen in the diagram at the right of FIG. 1 as the grey shaded area). The detection surfaces 3 can be seen from the sideview (right side of figure) and are slightly raised (<1 mm) above the top of the suction head 4.

It should be understood that any suitable means may be used to fix the electrodes and the wires in place in the suction head. One such means is adhesive, such as epoxy; many other means are known in the art and may be used.

FIG. 2A shows a photograph of the insert ion end of Probe 1 showing the suction head 4, electrodes 2, 3, connector arm 1, tubing 12 and wires 13. FIG. 2B shows a photograph of the distal end of Probe 1, showing tubing 12, wires 13, connector 16, fitting 17 housing stopcock 18 and syringe port 19, and syringe 22.

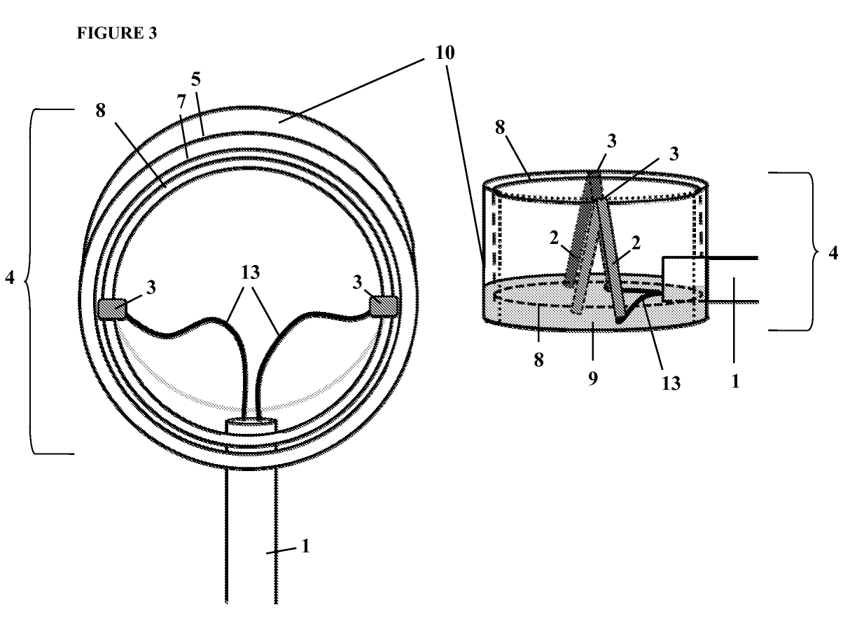

A second embodiment of the probe described herein is shown in FIG. 3 (hereinafter referred to as “Probe 2”). In this embodiment, the electrodes 2 are bent over an inner ring 8. The inner ring 8 is placed inside the wall 10 of the suction head. There are small detection surfaces 3, approximately 1 mm in length on opposite sides of the electrode head, seen at 3 o'clock and 9 o'clock positions (the connector arm 1 is at the 6 o'clock position; left side of figure). The bottom of the electrode is filled with epoxy 9 setting the inner ring 8, wires 13 and detecting surface 3 in place as seen in the diagram to the right (shown as the grey shaded area). The detection surfaces 3 can be seen from the side view (right side of figure) and are flush with the top of the suction head 4.

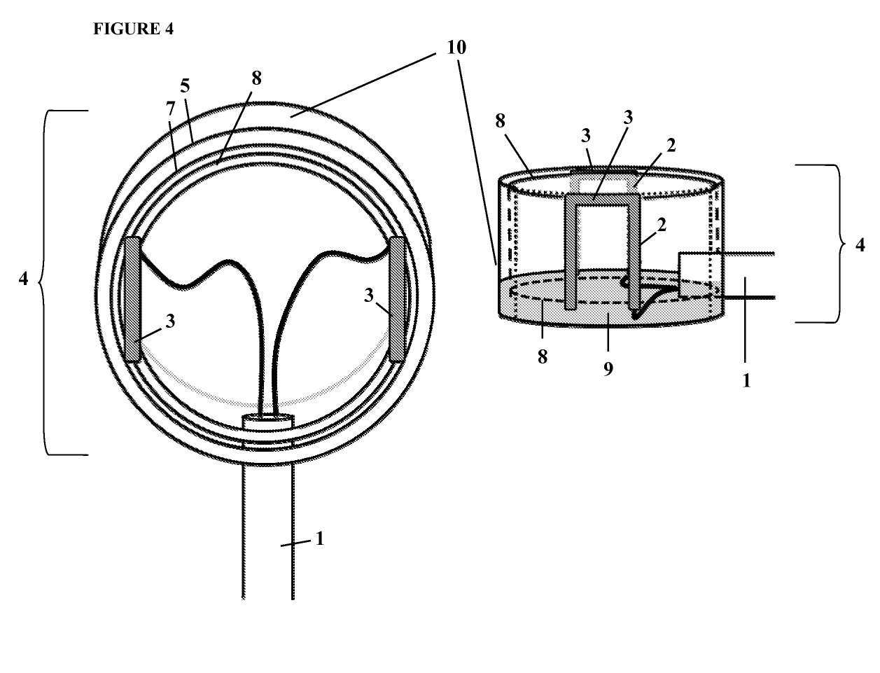

A third embodiment of the probe described herein is shown in FIG. 4 (hereinafter referred to as “Probe 3”). In this embodiment the electrodes 2 are bent over the inner ring 8 with flat detection surfaces 3, approximately 0.5 cm in length on opposite sides of the suction head 4, seen at 3 o'clock and 9 o'clock positions (left side of figure). The bottom of the electrode is filled with epoxy 9, setting the inner ring 8, wires and detecting surfaces in place as seen in the diagram to the right (grey shaded area). The detection surfaces 3 can be seen from the side view (right side of figure) and are flush with the top of the electrode.

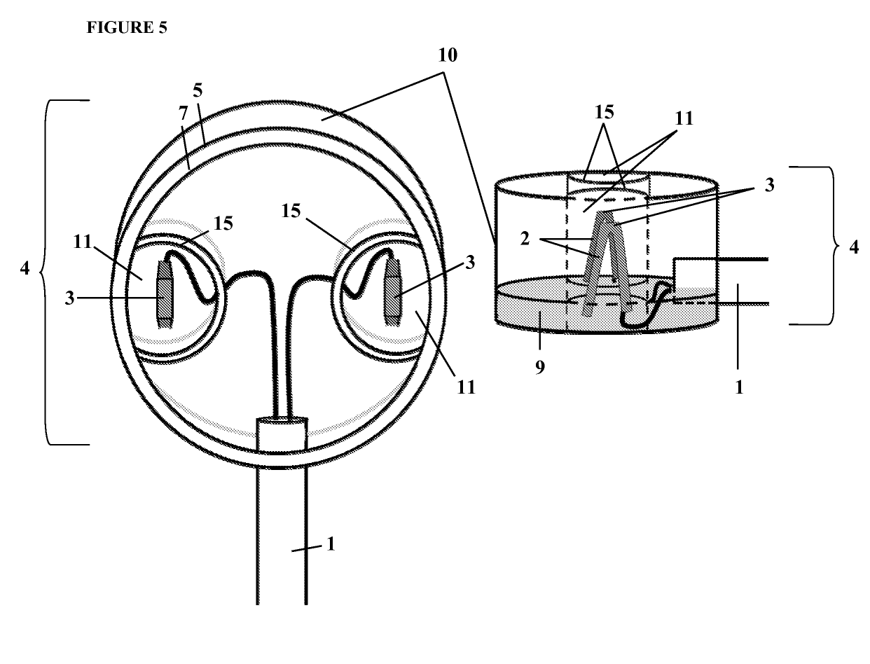

A fourth embodiment of the probe described herein is shown in FIG. 5 (hereinafter referred to as “Probe 4”). The probe shown in this embodiment also has a round suction head 4, this time 10 mm in diameter. The electrodes are still located at the 3 and 9 o'clock positions, but are now lowered or recessed by approximately 1 mm with respect to the top of the round suction head. Each electrode is encircled by a round fitting or wall 15 (approx. 3 mm in diameter) to form a well 11, whose edge is flush with the wall 10 of the suction head (See FIG. 5). Thus, the lowered or recessed electrodes are located within the wells. For this embodiment, conductive gel is injected into the electrode wells 11 prior to placing the insertion end of the probe on the skin or muscle, for example, prior to inserting the suction head/electrode into the vagina.

For Probes 1 to 4, the distal assembly of the probe remains the same as depicted in FIG. 6. In the embodiment shown in FIG. 6, the suction head 4 is connected to a catheter 12 of approximately 30 cm in length and in which the electrode leads 13 are located. The electrode leads are connected to any amplifier system using, e.g., alligator clips or any suitable fastening means. A schematic diagram of the suction heads 4 of Probes 1 to 4 is also shown in FIG. 6.

During use of probes with lowered or recessed electrodes, electrode paste may be injected into the circular region or well 11 surrounding each electrode 2.

In operation, a technician inserts the probe into the patient's lumen (e.g., vaginal opening, anus, mouth, nostril) and locates the desired location for EMG measurements. Once the desired location is identified, the insertion end of the probe is placed on this spot. In practice, the technician could locate the spot by inserting his/her fingertip and palpating to identify the desired location, or use a camera probe to identify the location. Once the probe is positioned in place, its correct positioning is verified holding the electrode against the mucous membrane wall and asking the patient to contract the muscle to be studied to verify the quality of the electrical signal. A syringe is then used to draw air from the probe, effectively creating a vacuum to hold the suction head in the correct position. When an adequate amount of suction has been created (e.g., approximately 1 cc of air in a syringe), the catheter is closed maintain the suction. For example, in an embodiment, a stopcock between the tubing and the syringe is closed. The technician can then remove his/her finger and the electrode will remain in the chosen location.

Thus in an embodiment the suction may be maintained by closing off a stopcock. With the stopcock closed, the syringe can be removed and the suction is maintained. It will be appreciated that other means of maintaining suction may be used and are meant to be encompassed by the present invention.

EMG measurements can then be taken using the probe. When the treatment session has concluded, the suction is released, e.g., the stopcock is opened, and the electrodes easily lift away from the tissue wall and the probe is withdrawn from the lumen.

In some embodiments, the probe is disposable.

In certain embodiments, the distal end apparatus (e.g., stopcock, fitting, connector) is sterilized and reused with a new catheter and a new insertion end of the probe.

Advantageously, with the probe suctioned onto the lumen walk measurements can be taken in a variety of postures or body positions and white the patient performs activities. Previously such measurements and biofeedback were measured mainly while the patient was lying down and if the patient sat up or stood up, if not held in place, previously known probes would move off target, and may even be expelled from the lumen. Using this probe, measurements may be performed while the patient performs functional activities such as sitting, standing, jumping, catching, throwing or running. A patient may even sneeze, laugh or cough white measurements are taken. It is unprecedented to be able to study what happens to the muscles during such incontinence-causing activities. Using EMG white the patent is undergoing activities is advantageous over currently known probes.

When the probe is in situ, it forms an appropriate differential electrode channel located over the muscle to be studied. In an embodiment, the electrodes are approximately 1 mm². It should be understood that the size of the electrodes can vary. In other embodiments, the electrodes are approximately 0.5 mm², 1.5 mm², 2 mm², 2.5 mm² or between about 0.5 mm² and about 2.5 mm². In an aspect, the small size of the electrodes and their small (e.g., 1 cm) inter electrode distance makes them less likely to record crosstalk than other electrodes currently available on the market. The orientation of the electrodes along the rim 5 results in the electrodes being located along the length of the pelvic floor muscles as is standard practice in EMG, but is not the case for most commercially available probe designs.

In an embodiment, the inter-electrode distance is about 7 mm, about 10 mm, between about 5 mm and about 12 mm, between about 7 mm and about 12 mm, between about 9 mm and about 12 mm, between about 7 mm and about 10 mm, or about 1 cm or less.

In an embodiment, the outer diameter of the opening formed by the suction head or the vessel is about 7 mm, about 10 mm, between about 5 mm and about 12 mm, between about 7 mm and about 12 mm, between about 9 mm and about 12 mm, between about 7 mm and about 10 mm, or about 1 cm or less. It will be understood that the inner diameter will vary depending on the thickness of the wall of the suction head and of the inner ring, if present. In an embodiment, the inner diameter is about 1/16 inch less than the outer diameter.

In an embodiment, the ails of the shaped portion are about 10 mm to about 12 mm high.

The probe of the invention is particularly advantageous for measuring muscles such as PFMs or intestinal muscles which require placement of the probe on a moist mucous membrane. The use of releasable suction allows the probe to adhere temporarily to a moist mucous membrane such as a vaginal wall or a large intestine wall. However the probe is also suitable and intended for use for EMG recordings and/or electrical stimulation at any specific and localized muscle accessible via a membrane of a body cavity. Non-limiting examples of body cavities where the probe may be used include the vagina, the rectum, the colon, the mouth, the nostril and/or the alimentary canal.

In some cases two or more probes may be inserted and used in the same lumen simultaneously. For example, two such probes can be attached one to each side of the vaginal wall in order to record activity from both the right and left pelvic floor muscles separately but simultaneously.

The probe of the invention has the potential to otter several distinct advantages over currently available probes. As noted above, in one aspect, it may be much less prone to recording crosstalk. The electrodes are carefully placed over the location of the muscles in each subject, so the electrode location matches the subject's anatomy. The probe may also be more comfortable for users as there is no large probe inserted. In other aspects, it may have the advantages of not changing the contractile properties of the muscle, and of not moving out of the area during tasks that increase pressure (e.g., moving out of the vagina when faced with increased intra-abdominal pressure).

In alternative embodiments, the probe described herein provides an opportunity to perform EMG recordings that are specific and localized to muscles that abut a moist cavity (vagina, rectum, mouth, esophagus etc), while minimizing crosstalk and motion artifact. The probe uses reversible suction to temporarily adhere the electrodes to a moist mucous membrane such as a vaginal wall, anal canal or mouth. The close relative position of the electrodes minimizes crosstalk, and adhesion of the electrodes to the tissues via suction prevents functional activities from causing probe movement and motion artifact. Located at the probe's first end, which is known herein as its “insertion end”, is a bowl-shaped portion. The bowl-shaped portion has a connector arm attached to it that is also attached to a length of flexible tubing (e.g., silicon tubing 30 cm length). The tubing should be strong enough to maintain some suction (i.e., vacuum) without collapsing. The bowl portion comprises walls that surround an opening (e.g., a round opening 1-2 cm in diameter). On the sides of the bowl, two wells house electrodes (e.g., conductive material such as stainless steel, gold, silver, platinum or silver-silver chloride, etc.), one on each side of the opening, which may be recessed into the wells. These wells and electrodes may be located at any position relative to the connector arm and length of the tubing. As an example, for recordings from the pelvic floor muscles, they are located in the 3 and 9 o'clock positions such that, when the probe is in situ, the electrodes are aligned parallel to the muscle fibers of the pelvic floor muscles. By recessing the electrodes within the wells, conductive gel or paste can be injected into the well before insertion, thus creating a more electrically stable interaction between the electrodes and the tissue membrane and thus reducing motion artifact contamination of the EMG recordings. Each electrode is operationally connected to an electrical wire that runs the length of the tubing and that is housed inside the central longitudinal cavity of the hollow tubing. The wires exit the tubing at its distal end and connect to a variety of pre-amplifier inputs (e.g., via snap fastener, alligator clip, etc.). The distal ends of the probe and of the tubing are the ends that are remote from the insertion end. Inserted in the distal end of the tubing is a hollow connector that has a longitudinal central cavity. The connector has a first end that is attached (e.g., frictionally connected) to the tubing. At the connector's second end it is attached to a fitting. The fitting has a hollow central longitudinal core that can be in an open position or a closed position, i.e., it can be reversibly closed off. In an embodiment, this reversible closing of the fitting is performed using a stopcock that is located at the side of the fitting in between its ends. At the fitting's distal end is a port that is suitable to receive a syringe. For example, the syringe may screw into the port or may be inserted using friction.

### EXAMPLES

The present invention will be more readily understood by referring to the following examples, which are provided to illustrate the invention and are not to be construed as limiting the scope thereof in any manner.

Unless defined otherwise or the context clearly dictates otherwise, all technical and scientific terms used herein have the same meaning as commonly understood by one of ordinary skill in the art to which this invention belongs. It should be understood that any methods and materials similar or equivalent to those described herein can be used in the practice or testing of the invention.

### Example 1

A study was performed to determine the reliability and validity of Probe 1 of the invention when recording surface EMG from the PFMs in healthy women. Probe 1 was also compared to a commonly used electrode (Femiscan™; surface area 1.75 cm² each). The Femiscan™ device was re-wired to record differential configurations from the right and left PFMs separately since this is a more appropriate way to record such muscle activation.

Reliability refers to between-trial reliability for the probe. Between-day reliability is not expected to be high for any EMG data since there is, among other factors, inherent variation in electrode position relative to active muscle fibers. Validity refers to the effect of the hip adductor (Add) and external rotator (ER) contractions on the signal recorded at the PFMs. In this case we were particularly interested in determining whether the recorded EMG signals come from the PFMs or represent crosstalk from nearby muscles.

Twenty healthy nulliparous women between 18 and 50 years of age participated in this study. Women were brought in for a training/familiarization session in which they were taught how to perform an isolated PPM contraction, and practiced the tasks to be asked of them on the evaluation day.

For the reliability testing, the women were asked to perform three repetitions of maximum voluntary contractions (MVC) of the PFMs.

For the validity/crosstalk testing, women were asked to perform either isolated hip contractions (Add/ER) or combined PPM and hip contractions (Add/ER).

For the isolated hip contractions the participants were asked to keep their PFMs relaxed while they performed hip muscle contractions at intensities of 25% MVC, 50% MVC, and MVC (i.e., the instruction was: “keeping your pelvic floor muscles relaxed, don't let me move your leg”). For the isolated hip contraction, provided that the PFMs were relaxed, any increases in EMU amplitude were likely due to crosstalk. One difficulty of these experiments is that the PFMs are thought to contract synergistically with the hip muscles, particularly at high intensity contra lions of the hip muscles, and therefore an increase in activity seen on the PPM EMG electrodes might represent co-activation or crosstalk, and the difference between these two is difficult to elucidate. In the case of this study, given that the same activities were performed with two different recording electrodes in situ (i.e., the Femiscan™ and Probe 1), if an increase in EMG activity at the PPM electrode was seen when the hip muscle contractions were performed with both electrodes in situ, then it was not possible to determine whether the EMG activity recorded by the PFM electrodes was due to coactivation or due to crosstalk. If, however, there was an increase in EMG activity seen during hip muscle contractions when one electrode was in situ, but not when the other was in situ, then this result suggested that the electrode that saw the activity was recording crosstalk.

For the combined PPM and hip contractions, women were asked to contract their PFMs maximally, hold the contraction, and then add on the hip contraction (25% MVC, 50% MVC or MVC). For combined contractions, if the PFMs were already contracted maximally, any increases in amplitude during the added hip task were likely due to crosstalk. It should be noted that one complication of this phase of the experiment was that many women have difficulty maximally contracting their PFMs, and therefore the same interpretation as above was employed. i.e., if the increase in activity recorded from both PFM electrodes was present when the hip contractions were added to the maximal PPM contraction, then it was not possible to tell whether the electrodes were picking up crosstalk or co-activation. If, however, only one electrode demonstrated an increase in PFM EMG activity and the other did not, it is likely that that electrode was picking up crosstalk.

For the reliability testing, the data were analyzed to determine the intraclass correlation coefficients and the coefficients of variation. For the intraclass correlation coefficients, the reliability coefficient typically ranges from 0 to 1; values closer to 1 are more desirable. For the coefficients of variation, which represent the spread of the data as a percentage of the average value, values closer to 0 are more desirable.

For the validity testing, the data were tested using a two-way repeated measures ANOVA (General Linear Model) and differences in EMG RMS amplitudes were recorded when the p-value for the test was less than 0.05. The electrode and the intensity of the hip contraction were included as factors in the analysis.

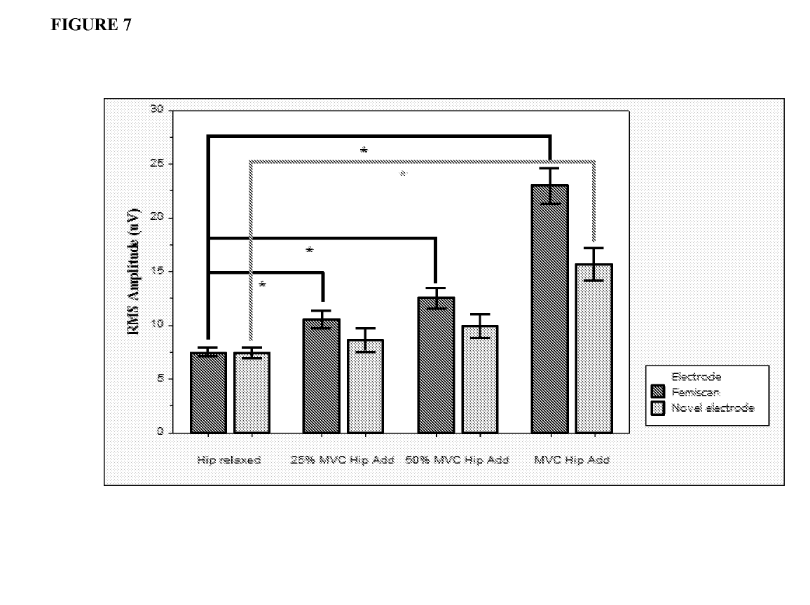

Results are shown in Table 1 below and in FIGS. 7-9.

TABLE 1
Between-trial reliability results.

| Task | Device | Side of muscle | ICC_{(3,1)} | CV (%) |
|---|---|---|---|---|
| MVC of PFMs | Femiscan ™ | Right | 0.943 | 11.2 |
| | | Left | 0.910 | 11.2 |
| | Probe 1 | Right | 0.964 | 8.6 |
| | | Left | 0.974 | 8.8 |

The effect of isolated hip adductor contractions on the EMG signal recorded at the PFMs is shown in FIG. 7. No significant differences between the electrodes were seen when the hip muscles were at rest. However, with a 25 or 50% MVC of the hip adductors, the EMG amplitude recorded by the Femiscan™ was increased significantly compared to when the hip adductors were relaxed, whereas for Probe 1, the EMG amplitudes recorded at 25 and 50% MVC weren't significantly different from the resting activity of the PFMs.

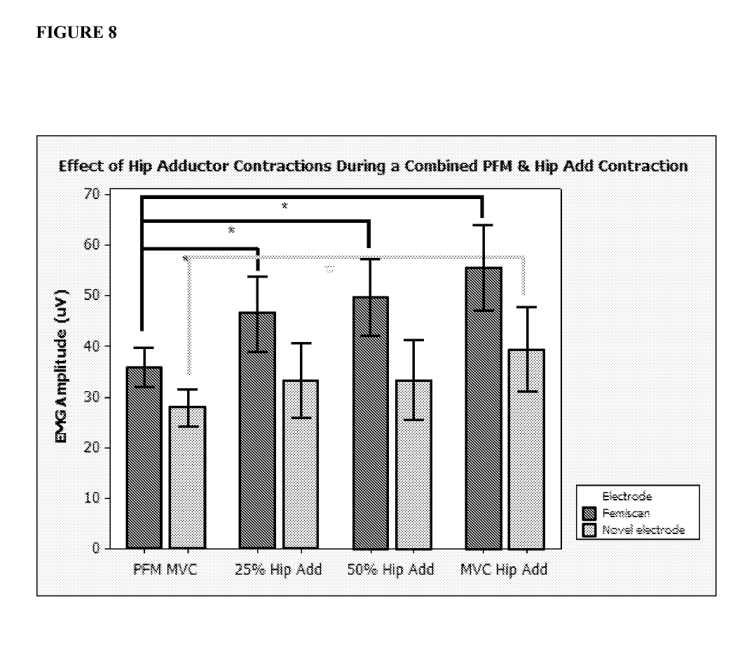

The effect of hip adductor contractions during a combined PFM and hip adductor contraction was similar and is shown in FIG. 8. The Femiscan™ recorded significantly higher EMG amplitudes during a combined PPM and hip adductor contraction, at 25%, 50%, and 100% hip intensities, compared to the EMG amplitude recorded during a PPM contraction alone. Probe 1, on the other hand, did not record significantly different EMG amplitudes during the 25% or 50% hip adduction tasks, compared to the amplitude recorded during a PFM MVC alone. The only significant increase in amplitude for probe 1 occurred during a hip adductor MVC, which means that at this intensity of hip muscle contraction, we could not determine whether the activity recorded from the PFM electrodes was related to crosstalk or co-activation.

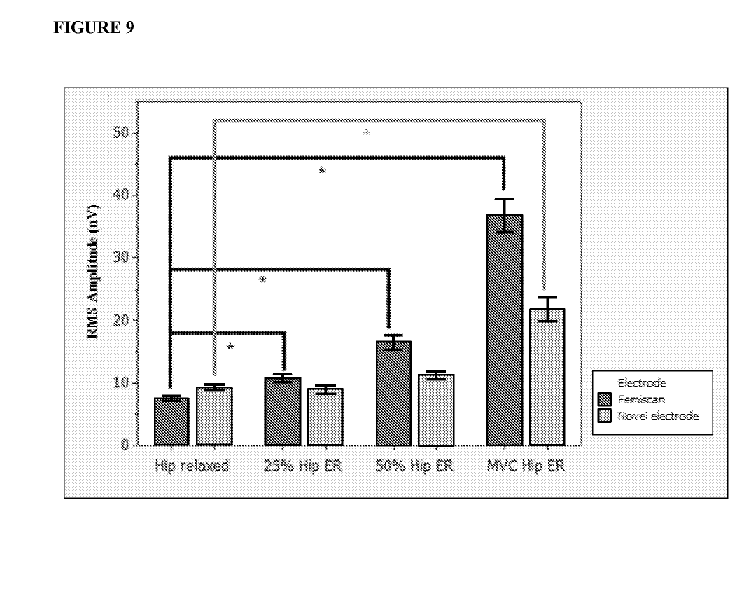

The effect of hip external rotation (ER) contractions alone or during a combined PFM produced the same results as the hip adductor contractions. The effect of hip ER contractions performed in isolation is shown in FIG. 9. During a PFM MVC alone, the vaginal electrodes recorded similar amplitudes from the PFMs. When adding on a 25%, 50% or 100% MVC hip ER contraction, the Femiscan™ recorded significantly higher amplitude compared to rest, whereas Probe 1 did not record significantly different amplitude compared to the rest values until a MVC of the hip external rotators was performed.

The results of this study show that probe 1 is as reliable within the same session as the Femiscan™. Advantageously, Probe 1 recorded less crosstalk from the hip adductors and external rotators than Femiscan™. It is noted that this was the first study to investigate the influence of obturator internus contractions on the signal recorded at the PFMs and that the study indicates that a significant improvement in crosstalk is obtained with probe 1 compared to the Femiscan™ electrode.

In sum, the study shows that Probe 1 is superior to the intravaginal Femiscan™ probe in terms of crosstalk, and is also reliable within the same session.

### Example 2

### Determination of Motion Artifact

A study was performed to determine whether EMG recordings made using the novel probe described herein have less motion artifact contamination than the Femiscan™ electrode, a commercially available electrode reconfigured to incorporate two differential EMG channels (one on each side of the vaginal wall) using stainless steel bars mounted on a cylindrical probe.

### Methods:

Eighteen healthy continent women with no signs of pelvic floor muscle dysfunction (such as urinary or fecal incontinence, pelvic pain disorders, or low back pain) were recruited from the Kingston (Ontario, Canada) community. Each participant performed ten repetitions of a maximal effort coughing task in the standing position with both the Femiscan™ probe and Probe 1 of the invention (see FIG. 1) in situ, with the probes tested in random order. EMU data were recorded from both sides of the vaginal wall using Delsys™ AMT-8 pre-amplifiers (bandwidth 20-450 Hz, input impedance >100 MOhm, common mode rejection ratio >120 dB at 60 Hz, Gain ×1000) at a sampling rate of 1000 Hz.

A second group of 15 women with stress urinary incontinence was recruited from the Kingston (Ontario, Canada) community. Each participant performed nine repetitions of the same coughing task with Probe 4 of the invention (see FIG. 5) in situ. The EMG instrumentation and data collection parameters did not differ between the groups.

The resultant dataset (924 raw EMG data files) was inspected for the presence of motion artifact; the dataset included 328 files from the Femiscan™ probe, 340 files from Probe 1, and 256 files from Probe 4.

Each EMG data file was notch filtered with a 5th order Butterworth filter, with corner frequencies at 58 and 62 Hz. Since motion artifact can be defined by the presence of a burst of low frequency activity that deviates from baseline EMG and lasts longer than 5 milliseconds (Konrad, P., 2005, The ABC of EMG: A practical introduction to kinesiological electromyography [PDF document], retrieved from http://www.noraxon.com/downloads/educational.php3), and by spectral frequencies in the 0-20 Hz range (De Luca, C., 2002, Surface electromyography: Detection and recording [PDF document], retrieved from http://www.delsys.com/KnowledgeCenter/Tutorials_Technical%20Notes.html), in order to determine whether a file was contaminated with motion artifact, two criteria had to be met: i) a peak spectral density in the 0-20 Hz range that was greater than the peak found in the 20-250 Hz range; and ii) visual inspection of a shift in EMG signal away from baseline that lasted at least 5 ms in duration.

Z-ratios were calculated to determine whether there were significant differences between the proportion of files containing motion artifact when the coughing task was performed with the Femiscan™ electrode, Probe 1 or Probe 4 in situ.

### Results:

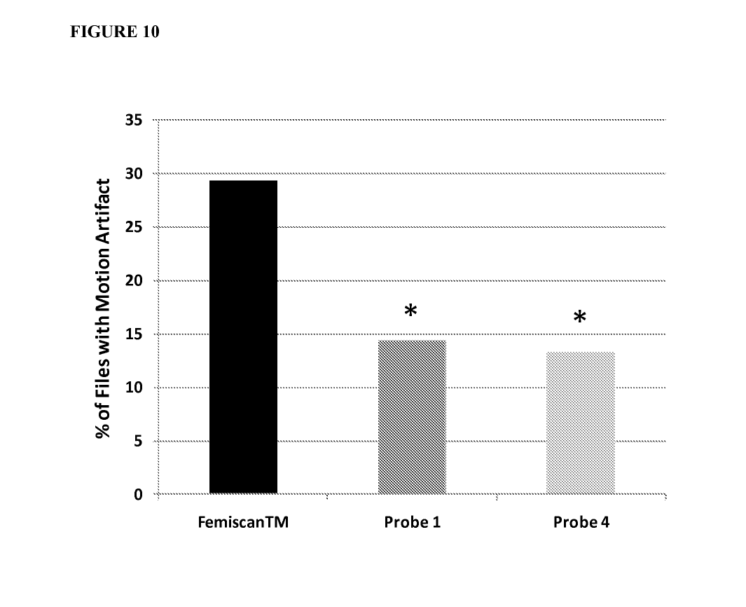

The Femiscan™ electrode generated a significantly greater proportion of files contaminated with motion artifact than either Probe 1 (z=4.66, p<0.0002) or Probe 4 (z=4.62, p<0.0002). Of the coughs recorded with the Femiscan™ electrode, 29.3% ( 96/328) were contaminated with motion artifact whereas only 14.4% ( 49/340) of those recorded with Probe 1 (see FIG. 10) and 13.3% ( 34/256) of the coughs recorded with Probe 4 were contaminated by motion artifact. There was no significant difference in proportion of files contaminated by motion artifact between Probes 1 and 4 of the invention.

These results show that the probes of the invention provide a significant improvement over the Femiscan™ commercially available vaginal electrode probe in terms of motion artifact contamination of the recorded signals. Both the probe with recessed electrodes (electrodes located below the top of the suction head assembly) housed in separate wells at the approximately 3 and 9 o'clock positions (Probe 4) and the probe with the electrode wires bent over the suction head and the electrode tips located flush with the suction head edge (Probe 1) provided similar improvement in terms of motion artifact.

Motion artifact occurs when there is motion of the electrode across the skin (or membrane) surface, when the muscle moves relative to the location of the electrodes, or when there is motion of the leads that connect the electrodes to the recording system. The results indicate that the probe of the invention can hold the electrodes solidly in place, thus minimizing motion artifact. Motion artifact cannot be expected to be eliminated completely since the suction head does not prevent motion of the muscle relative to the skin surface, or motion of the leads or wires.

### Example 3

### Determination of Crosstalk

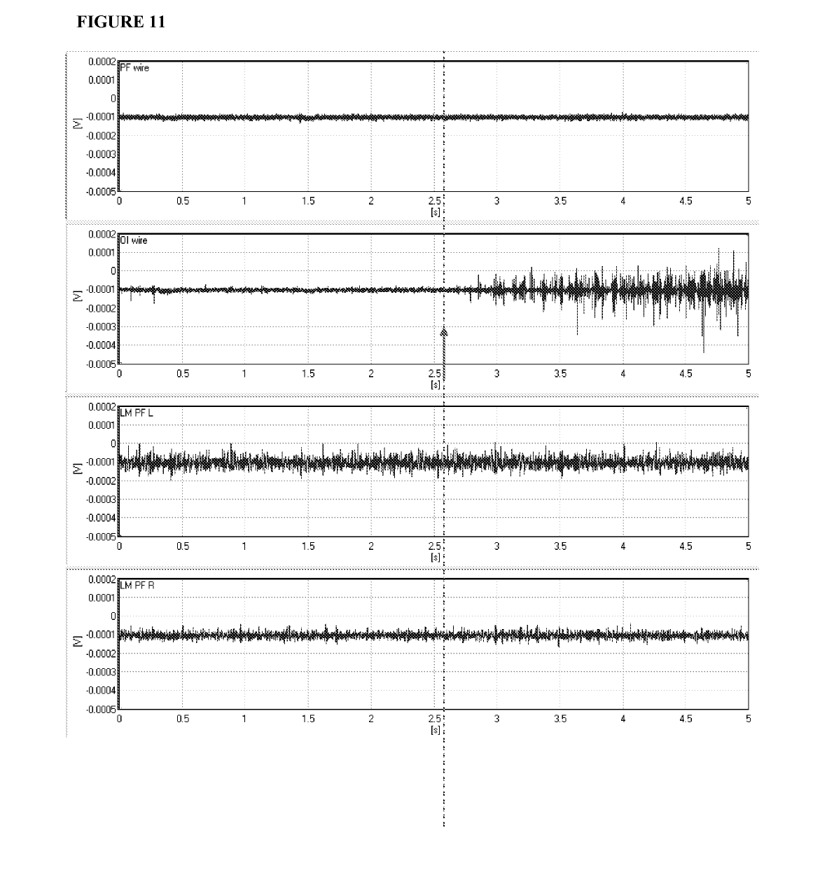

A study was performed on three volunteers (healthy, nulliparous women) to determine whether EMG recordings made using Probe 4 have crosstalk contamination from the obturator internus muscle (Exemplar data are presented in FIG. 1). EMG data were recorded from PFMs during a contraction of the hip external rotators, which should elicit obturator internus activity but not pelvic floor muscle activity. The following data were recorded: pelvic floor muscle EMG data were recorded using fine wire electrodes located in the right pelvic floor muscle (gold standard) (top panel of FIG. 11); obturator internus EMG data were recorded using fine wire electrodes placed in the right obturator internus muscle (second panel from top in FIG. 11); pelvic floor muscle EMG data were recorded simultaneously using Probe 4 (bottom two panels in FIG. 11; third panel from top shows data recorded with the probe located on the left side of the vagina, and the bottom panel shows data recorded with the probe located on the right side of the vagina). The arrow in FIG. 11 indicates the onset of obturator internus muscle activity during the hip external rotation contraction.

There were several tasks during which here was EMG activity recorded from the fine wire electrodes inserted into the obturator internus muscle, hut no activity recorded on either the fine wire or Probe 4 electrodes located in or over the PFMs. As an example, the fine wire EMG data shown in the top panel of FIG. 11 indicates that the right pelvic floor muscle remains quiet while the obturator internus muscle contracts. The bottom two panels show that there is no EMG activity recorded from the PFMs by Probe 4 during contraction of the obturator internus muscle and no crosstalk recorded from the obturator internus by Probe 4.

While specific embodiments of the present invention have been described in the examples, it is apparent that modifications and adaptations of the present invention will occur to those skilled in the art. The embodiments of the present invention are not intended to be restricted by the examples. It is to be expressly understood that such modifications and adaptations which wilt occur to those skilled in the art are within the scope of the present invention, as set forth in the following claims. For instance, features illustrated or described as part of one embodiment can be used in another embodiment, to yield a still further embodiment. Thus, it is intended that the present invention cover such modifications and variations as come within the scope of the claims and their equivalents.

The contents of all documents and references cited herein are hereby incorporated by reference in their entirety.

1. A probe for electromyography, comprising:
(a) an insertion end for attachment to a membrane, the insertion end comprising:
(i) a shaped portion which forms a vessel open at the top;
(ii) at least two electrodes attached to the shaped portion; and
(iii) at least two wires, each wire connected at a first end to a said electrode and suitable for connection to an electronic device at a second end; and
(b) a distal end for connection to a means for providing suction and for attaching the at least two wires to an amplifier system;
wherein the insertion end is attachable via suction to the membrane such that the electrodes contact the membrane and an electromyography signal is recorded from muscles accessible via the membrane.

2. The probe of claim 1, wherein the insertion end further comprises a connector arm for attachment to a catheter, the connector arm being attached to the shaped portion.

3. The probe of claim 1, wherein the shaped portion is round, substantially round, oval or substantially oval.

4. The probe of claim 1, wherein the vessel is bowl-shaped.

5. The probe of claim 1, wherein the at least two electrodes are disposed substantially at or on walls of the shaped portion.

6. The probe of claim 2, wherein the connector arm is connected to the catheter at a first end, and the at least two wires are housed inside the central longitudinal cavity of the catheter and exit the catheter at a second end.

7. The probe of claim 6, wherein the second end of the catheter is attached to a means for providing suction.

8. The probe of claim 7, wherein the means for providing suction is a syringe or a pump.

9. (canceled)

10. The probe of claim 6, wherein the second end of the catheter is attached to a first end of a hollow connector having a longitudinal central cavity, and a second end of the hollow connector is attached to a means for providing suction.

11. (canceled)

12. (canceled)

13. The probe of claim 10, wherein the second end of the hollow connector is attached to a first end of a fitting having a hollow central longitudinal core that can be in an open or a closed position, and a second end of the fitting is attached to a means for providing suction.

14. The probe of claim 13, wherein the fitting is a stopcock.

15. The probe of claim 1, wherein the at least two electrodes are bent over the wall of the shaped portion, located within the top of the wall of the shaped portion, encircled by a round fitting attached to the wall of the shaped portion, located at or near the top of the wall of the shaped portion, or attached to or bent over an inner ring inside the wall of the shaped portion.

16. (canceled)

17. (canceled)

18. (canceled)

19. The probe of claim 15, wherein the inner ring is fixed in place using an adhesive.

20. (canceled)

21. The probe of claim 1, wherein the at least two electrodes are located below the top of the wall of the shaped portion.

22. The probe of claim 21, wherein the at least two electrodes are encircled by a round fitting attached to the wall of the shaped portion.

23. The probe of claim 21, wherein the at least two electrodes are located at about 1 mm, or between about 0.5 mm to about 3 mm, below the top of the wall of the shaped portion.

24. The probe of claim 1, wherein the outer diameter of the shaped portion is about 7 mm, about 10 mm, or between about 7 mm and about 12 mm.

25. The probe of claim 1, wherein the walls of the shaped portion are about 10 mm to about 12 mm high.

26. The probe of claim 1, wherein the membrane is in the vagina and the muscles are pelvic floor muscles.

27. The probe of claim 1, wherein the membrane is in the rectum, the colon, the mouth, the nostril or the alimentary canal.

28. A probe for electromyography, comprising:
(a) an insertion end for attachment to a membrane, the insertion end comprising:
(i) a shaped portion which forms a bowl-shaped vessel open at the top and having a diameter of about 10 mm;
(ii) at least two electrodes attached to the shaped portion, wherein the at least two electrodes are encircled by a round fitting whose edge is flush with the wall of the shaped portion, and the electrodes are located at about 1 mm below the top of the wall of the shaped portion;
(iii) at least two wires, each wire connected at a first end to a said electrode and suitable for connection to an electronic device at a second end; and
(iv) a connector arm for attachment to a catheter, the connector arm being attached to the shaped portion, wherein the connector arm is at approximately the 6 o'clock position and the at least two electrodes are at approximately the 3 and 9 o'clock positions; and
(b) a distal end for connection to a means for providing suction and for attaching the at least two wires to an amplifier system;
wherein the connector arm is connected to the catheter at a first end, and the at least two wires are housed inside the central longitudinal cavity of the catheter and exit the catheter at a second end;
wherein the distal end comprises the second end of the catheter, which is attached to a first end of a hollow connector having a longitudinal central cavity, and a second end of the hollow connector is attached to a stopcock;
wherein the stopcock is attached to the means for providing suction;
wherein the insertion end is attachable via suction to the membrane such that the electrodes contact the membrane and an electromyography signal is recorded from muscles accessible via the membrane; and
wherein the means for providing suction is a syringe or pump.

29. The probe of claim 1, wherein the distance between the at least two electrodes is between about 7 mm and about 10 mm.

30. A method for performing electromyography, comprising:
(a) placing the probe of claim 1 at a location for electromyographical study;
(b) applying suction so that the insertion end attaches to a membrane;
(c) attaching the wires to an amplifier system; and
(d) measuring an electromyography signal.

31. (canceled)

32. The method of claim 30, wherein once suction is applied and maintained, the insertion end is substantially fixed at a position on the membrane.

33. The method of claim 30, wherein electromyography is conducted in respect of one or more muscles accessible via the membrane of a body cavity.

34. The method of claim 33, wherein the body cavity is the vagina.

35. The method of claim 33, wherein electromyography is conducted in respect of pelvic floor muscles.

36. The method of claim 34, wherein the electrodes are aligned along the anteroposterior axis of a subject when the insertion end is attached to the membrane.

37. The method of claim 33, wherein the body cavity is the rectum, the colon, the mouth, the nostril or the alimentary canal.

38. The method of claim 30, wherein, when the electrodes are located below the top of the wall of the shaped portion, conductive paste is applied to the electrodes before the probe is placed.

39. A method for performing electromyography of pelvic floor muscles in a subject, comprising:
(a) placing the probe of claim 1 on a vaginal membrane;
(b) applying suction so that the insertion end attaches to the membrane;
(c) attaching the wires to an amplifier system; and
(d) measuring an electromyography signal;
wherein the insertion end is attached to the membrane such that the electrodes are aligned along the anteroposterior axis of the subject.

40. The probe of claim 1, wherein the probe is disposable and/or sterilizable.

41. (canceled)
